(19) United States
(12) Patent Application Publication
(10) Pub. No.: US 2025/0319305 A1
(43) Pub. Date: Oct. 16, 2025

(54) TRANSDERMAL CURRENT-CARRYING PATCH

(71) Applicant: KIKKOMAN CORPORATION, Noda-shi, Chiba (JP)

(72) Inventor: Yosuke MASAKARI, Noda-shi, Chiba (JP)

(73) Assignee: KIKKOMAN CORPORATION, Noda-shi, Chiba (JP)

(21) Appl. No.: 18/870,027

(22) PCT Filed: May 24, 2023
(86) PCT No.: PCT/JP2023/019349

§ 371 Date: Nov. 27, 2024

(30) Foreign Application Priority Data
May 30, 2022 (JP) 2022-087996

(51) Int. Cl. A61N 1/20

(52) U.S. Cl. CPC A61N 1/20

(57) ABSTRACT

A transdermal current-carrying patch 1 includes an electrode body 10 having an anode electrode 11 and a cathode electrode 12, and conductive portions 20A and 20B disposed to come into contact with the electrode body 10. In the transdermal current-carrying patch 1, the anode electrode 11 and the cathode electrode 12 are brought into contact with a target part of a subject via the conductive portions 20A and 20B to form an electric circuit that generates a weak current to flow through the target part. The weak current flowing through the living body by the electric circuit is a DC current having a current density of 0.5 μA/cm² or more and less than 500 μA/cm².

### TECHNICAL FIELD

The present invention relates to a transdermal current-carrying patch.

### BACKGROUND ART

Patent Literatures 1 to 3 disclose examples of various current-carrying patches capable of applying current-carrying stimulation.

### CITATION LIST

### Patent Literature

Patent Literature 1: Japanese Unexamined Patent Publication No. 2016-144634
Patent Literature 2: Japanese Unexamined Patent Publication No. 2016-067401
Patent Literature 3: Japanese Unexamined Patent Publication No. 2021-115330

### SUMMARY OF INVENTION

### Technical Problem

Patent Literature 1 discloses a current-carrying patch that generates a very small amount of current to flow through a living body. In this current-carrying patch, it has been confirmed by an experiment that a very small amount of current of, for example, 0.1 μA to 2 μA or 4 μA to 5 μA can flow (see paragraphs 0029 and 0030 and the like of Patent Literature 1), and a current density of the current flowing in this current-carrying patch is, for example, smaller than 0.5 μA/cm² according to a simulated experiment to be described later. Patent Literature 1 proposes that such a current-carrying patch is used for treatment, but an improving effect when the current-carrying patch is used for treatment has not been verified, and the improving effect is unknown. However, there is a demand for improving a target part in a living body (for example, pain alleviation) by using a small therapeutic tool such as a current-carrying patch, and it is desired to provide such a current-carrying patch.

An object of the present invention is to provide a transdermal current-carrying patch capable of improving remedial effect on a target part.

### Solution to Problem

(1) As one aspect, the present invention relates to a transdermal current-carrying patch. The transdermal current-carrying patch includes a positive electrode, a negative electrode, and a conductive portion disposed to come into contact with the positive electrode and the negative electrode to correspond to the positive electrode and the negative electrode. In this transdermal current-carrying patch, an electric circuit that generates a weak current to flow through a living body by bringing the positive electrode and the negative electrode into contact with the living body via a conductive portion is formed. The weak current flowing through the living body by the electric circuit is a DC current having a current density of 0.5 μA/cm² or more and less than 500 μA/cm².

In this transdermal current-carrying patch, the electric circuit that generates the weak current to flow through the living body by bringing the positive electrode and the negative electrode into contact with the living body via the conductive portion is formed, and the weak current flowing through the living body by the electric circuit is the DC current having the current density of 0.5 μA/cm² or more and less than 500 μA/cm². According to findings by the present inventor, it has been found that the remedial effect on the target part can be significantly improved by setting the weak current flowing through the living body to the DC current of 0.5 μA/cm² or more which is higher than a very small amount of current (for example, 0.2 μA/cm²). Accordingly, according to this transdermal current-carrying patch, the remedial effect of the target part can be improved. In addition, when the current density of the current flowing through the living body is 500 μA/cm² or more, the user may feel stimulation. Thus, in the transdermal current-carrying patch, the electric circuit is formed such that the current density of the current flowing through the living body is less than 500 μA/cm². Accordingly, the transdermal current-carrying patch can be used for a long period of time (for example, can be attached to a predetermined part of the user), and the remedial effect of the target part can be further improved.

(2) In the transdermal current-carrying patch of the above (1), the electric circuit is preferably configured to generate a DC current having a current density of 10 μA/cm² or more to flow when the electric circuit is connected to a resistor of 5Ω. In this case, the remedial effect of the target part can be more reliably improved.

(3) In the transdermal current-carrying patch of the above (1) or (2), the electric circuit is preferably configured to generate a DC current having a current density of 35 μA/cm² or more to flow when the electric circuit is connected to a resistor of 5Ω. In this case, the remedial effect of the target part can be further improved.

(4) In the transdermal current-carrying patch according to any one of the above (1) to (3), the electric circuit is preferably configured to generate a DC current having a current density of 60 μA/cm² or more to flow when the electric circuit is connected to a resistor of 5 kΩ. In this case, the remedial effect of the target part can be further improved.

(5) In the transdermal current-carrying patch according to any one of the above (1) to (4), the electric circuit is preferably configured to generate a DC current having a current density of less than 500 μA/cm² to flow when the electric circuit is connected to a resistor of 1 kΩ. In this case, it is possible to prevent the user from feeling stimulation regardless of the condition of the skin, and it is possible to more reliably use the transdermal current-carrying patch for a long period of time. Accordingly, it is possible to further improve the remedial effect of the target part.

(6) In the transdermal current-carrying patch according to any one of the above (1) to (5), the electric circuit may be configured such that a current density of a weak current flowing at a point in time when 10 minutes elapses at the latest after the electric circuit is connected to a resistor of 5 kΩ is 10 μA/cm² or more and 175 μA/cm² or less. In this case, it is possible to continuously improve the remedial effect of the target part by attaching the transdermal current-carrying patch to the target part for a long period of time.

(7) In the transdermal current-carrying patch according to any one of the above (1) to (6), the electric circuit is preferably configured to generate a DC current having a current density of 10 μA/cm² or more and 30 μA/cm² or less to flow when the electric circuit is connected to a resistor of 10 kΩ. In this case, the remedial effect of the target part can be further improved.

(8) In the transdermal current-carrying patch according to any one of the above (1) to (7), the electric circuit is preferably configured to generate the amount of energy of 50 mJ or more in the electric circuit when the electric circuit is connected to a resistor of 10 kΩ. In this case, the remedial effect of the target part can be further improved.

(9) Preferably, the transdermal current-carrying patch according to any one of the above (1) to (8) further includes a connection portion configured to electrically connect the positive electrode and the negative electrode to each other, the conductive portion includes a plurality of conductive portions, respectively, corresponding to the positive electrode and the negative electrode, each of the plurality of conductive portions includes a sponge having an air bubble and a buffer agent made of an electrolyte, a solid of the buffer agent is exposed on an inner wall surface of the air bubble, and at least one electrode of the positive electrode and the negative electrode carries an enzyme that catalyzes an oxidation-reduction reaction. In this case, the electron transfer mediator is preferably fixed to the electrode that carries the enzyme, and the electron transfer mediator is more preferably a mediator of a quinone-based compound or a phenylenediamine-based compound. According to such a configuration, it is possible to more reliably realize the setting of the weak current flowing through the living body to any of the above-described ranges, and to more reliably improve the remedial effect of the target part.

(10) In the transdermal current-carrying patch according to any one of the above (1) to (9), an area of each of the positive electrode and the negative electrode may be 80 cm² or less. In this case, the transdermal current-carrying patch can be downsized, and the transdermal current-carrying patch can be easily attached to the target part of the user for a long period of time. Accordingly, the remedial effect of the target part can be further improved.

(11) Preferably, the transdermal current-carrying patch of the above (9) further includes a double-sided adhesive tape that has openings provided to house the positive electrode and the negative electrode, and has insulating properties, the connection portion is fixed to one surface of the double-sided adhesive tape, and the conductive portion is fixed to the other surface of the double-sided adhesive tape. In this case, it is possible to downsize the transdermal current-carrying patch while securing both fixation of the positions of the positive electrode, the negative electrode, and the conductive portion and ion insulation between the plurality of conductive portions.

(12) As another aspect, the present invention relates to an operation method of a transdermal current-carrying patch or a treatment method using a transdermal current-carrying patch. In this operation method or treatment method, a weak current flows through a living body by using the transdermal current-carrying patch of any one of the above (1) to (11). Such an operation or treatment can improve the remedial effect of the target part.

### Advantageous Effects of Invention

According to the present invention, it is possible to improve the remedial effect of the target part.

### BRIEF DESCRIPTION OF DRAWINGS

FIG. 1 is an exploded perspective view of a transdermal current-carrying patch according to an embodiment of the present invention.

FIG. 2 is a schematic view illustrating a relationship between a catalyst and an electron transfer mediator at an anode electrode of the transdermal current-carrying patch illustrated in FIG. 1.

FIG. 3 is a graph representing a current density flowing through a living body by the transdermal current-carrying patch illustrated in FIG. 1.

FIG. 4 is a graph representing an example of a current density of the transdermal current-carrying patch illustrated in FIG. 1.

FIG. 5 is a graph representing results of evaluating delayed onset muscle soreness using the transdermal current-carrying patch.

FIG. 6 is a graph representing results of evaluating exercise performance using the transdermal current-carrying patch.

FIG. 7 is a graph representing results of evaluating resolution of stiff shoulder using the transdermal current-carrying patch.

FIG. 8 is a graph representing an example of results of evaluating resolution of temporomandibular joint disorder using the transdermal current-carrying patch.

FIG. 9 is a graph representing another example of results of evaluating the resolution of the temporomandibular joint disorder using the transdermal current-carrying patch.

FIG. 10 is a diagram schematically illustrating a patch experiment method of a comparative example.

### DESCRIPTION OF EMBODIMENTS

Hereinafter, a transdermal current-carrying patch according to an embodiment of the present invention will be described in detail with reference to the drawings. In the description, the same elements or elements having the same functions will be assigned the same reference signs, and redundant description will be omitted.

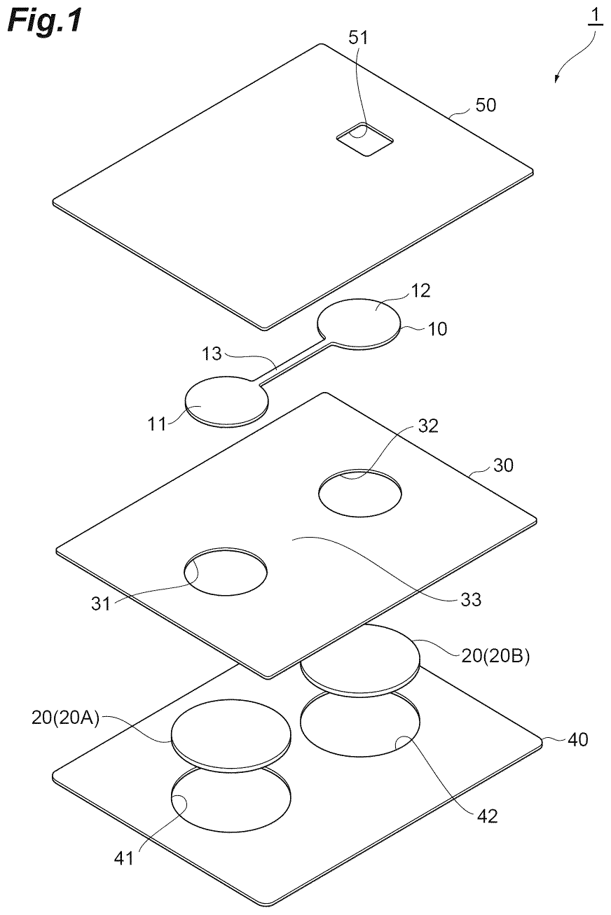

FIG. 1 is an exploded perspective view of the transdermal current-carrying patch according to the embodiment of the present invention. A transdermal current-carrying patch 1 is a current patch using a biobattery using an enzyme, and includes an electrode body 10 (a plurality of electrodes), two conductive portions 20 (conductive layers or a plurality of conductive portions), an adhesive layer 30, a separator 40, and a surface film 50 as illustrated in FIG. 1. In use, the transdermal current-carrying patch 1 is used by removing the separator 40 and being attached to a skin (living body) of any part (for example, shoulder, arm, or jaw) of a body of a subject (user) with the adhesive layer 30. As will be described in detail later, by such attaching, in the transdermal current-carrying patch 1, each electrode of the electrode body 10 comes into contact with the part of the subject via the conductive portions 20 to form an electric circuit that generates a weak current to flow. In the present embodiment, in the electric circuit, a weak current flowing through the part of the subject and a proximity region thereof is, for example, a DC current having a current density of 0.5 μA/cm² or more and less than 500 μA/cm², and is a slightly stronger current than an extremely weak current. However, the transdermal current-carrying patch 1 is set to generate a current weaker than a current density of 500 μA/cm², which is a guide for the subject to feel stimulation. Note that, the weak current flowing through the part or the like of the subject by the transdermal current-carrying patch 1 may be 1 μA/cm² or more.

The electrode body 10 includes an anode electrode 11 (negative electrode), a cathode electrode 12 (positive electrode), and a lead 13 (connection portion). The lead 13 connects the anode electrode 11 and the cathode electrode 12. The anode electrode 11, the lead 13, and the cathode electrode 12 may be disposed in this order, and may be formed as an integrated member. The electrode body 10 has a thickness of, for example, about 0.1 mm to 2.0 mm. A size of the transdermal current-carrying patch 1 is preferably, for example, 1 cm to 10 cm in width and 1 cm to 10 cm in length. A size (area) of the electrode body 10 in the transdermal current-carrying patch 1 may be smaller than a size of the entire transdermal current-carrying patch 1, and sizes (areas) of the anode electrode 11 and the cathode electrode 12 may be appropriately modified as a geometric surface area according to a part to be attached or a range in which a weak current is desired to flow, and may be, for example, 80 cm² or less, 50 cm² or less, 40 cm² or less, 30 cm² or less, 20 cm² or less, 10 cm² or less, 1 cm² or less, 0.5 cm² or less, and 0.1 cm² or less. Such a small transdermal current-carrying patch 1 may be attached to a pain part, or a plurality of transdermal current-carrying patches 1 may be attached to the pain part. In addition, the transdermal current-carrying patch 1 may have a configuration in which one electrode body 10 is disposed, or may have a configuration in which two or more electrode bodies 10 are disposed. Note that, a shape of the transdermal current-carrying patch 1 may be any shape such as a polygon, a pentagon, a quadrangle, a triangle, and a circle.

Examples of materials of the anode electrode 11, the cathode electrode 12, and the lead 13 include carbon materials such as carbon nanotubes, Ketjen black (registered trademark), glassy carbon (registered trademark), graphene, fullerene, carbon fiber, carbon fabric, and carbon aerogel; conductive polymers such as polyaniline, polyacetylene, polypyrrole, poly(p-phenylenevinylene), polythiophene, or poly(p-phenylenesulfide); semiconductors such as silicone, germanium, indium tin oxide (ITO), titanium oxide, copper oxide, and silver oxide; and metals such as gold, platinum, titanium, aluminum, tungsten, copper, silver, zinc, magnesium, iron, and palladium. In particular, from the viewpoint of flexibility and electrochemical stability, carbon materials such as carbon fabric or carbon nanotubes is preferable as a material of the electrode body 10. In particular, in a case where the enzyme is fixed to the electrode at a high density, the material of the electrode body 10 is preferably a carbon fabric modified with carbon nanotubes.

A catalyst that catalyzes an oxidation reaction may be carried on the anode electrode 11. Examples of such a catalyst include oxidoreductases such as glucose oxidase, glucose dehydrogenase (GDH), D-fructose dehydrogenase (FDH), alcohol oxidase, alcohol dehydrogenase, lactate oxidase, and lactate dehydrogenase. In addition to the enzyme, an electrode including one or more of magnesium and an alloy containing magnesium, aluminum and an alloy containing aluminum, calcium, iron, zinc, and the like may be used.

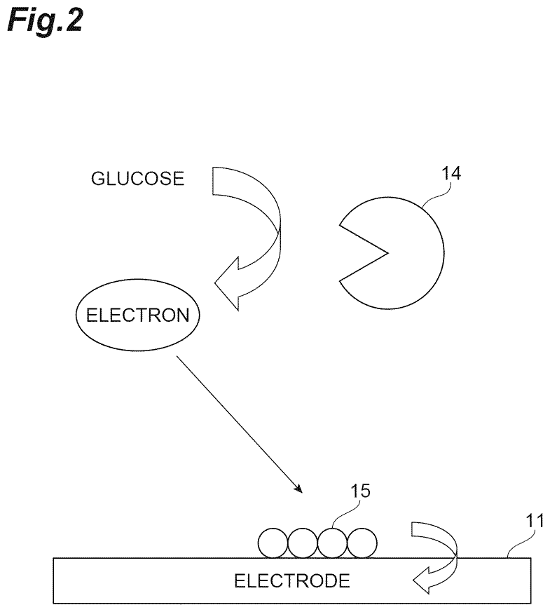

In addition, as illustrated in FIG. 2, an electron transfer mediator 15 that promotes electron movement between the electrode (anode electrode 11) and the enzyme 14 functioning as the catalyst in the biobattery is fixed to the anode electrode 11. In the anode electrode 11, for example, electrons can be efficiently extracted from glucose, which is a fuel, by the enzyme 14 and the electron transfer mediator 15 fixed to the electrode. Various electron transfer mediators may be used as the electron transfer mediator 15 used herein. Examples thereof include phenazines, viologens, cytochromes (for example, cytochrome b and cytochrome c), phenoxazines, phenothiazines, ferricyanides, for example, potassium ferricyanide, ferredoxins, ferrocenes, and osmium complexes, and derivatives thereof, and examples of a phenazine-based compound include mediators such as phenazine methosulfate (PMS), methoxy PMS, quinone-based compounds, and phenylenediamine-based compounds, but are not limited thereto. Examples of the quinone-based compound used for the mediator preferably include 1,4-naphthoquinone, 1,2-naphthoquinone, and 2-methyl-1,4-naphthoquinone. Examples of the phenylenediamine-based compound include N-isopropyl-N′-phenyl-p-phenylenediamine (IPPD), N,N′-diphenyl-p-phenylenediamine (DPPD), and N-(1,3-dimethylbutyl)-N′-phenyl-p-phenylenediamine (6PPD). By using such an electron transfer mediator, the current of the electric circuit when the transdermal current-carrying patch 1 is attached to a predetermined part of the subject can be increased to the above-described range.

A catalyst that catalyzes a reduction reaction is carried on the cathode electrode 12. Examples of such a catalyst include enzymes such as bilirubin oxidase (BOD), laccase, Cu efflux oxidase (Cueo), and ascorbic acid oxidase; transition metal complexes such as iron (II) phthalocyanine; and at least one metal of platinum, titanium, nickel, stainless steel, iron, manganese, zinc, copper, and molybdenum, or at least one metal oxide of at least one metal of calcium, iron, manganese, zinc, copper, and molybdenum.

The conductive portions 20 are water absorbents each disposed to come into surface contact with the anode electrode 11 and the cathode electrode 12. The conductive portion 20 has a structure in which dried fuel or electrolyte is contained inside a sponge. A conductive portion 20A coming into contact with the anode electrode 11 contains a fuel such as an organic matter that causes an oxidation reaction at the anode electrode 11. Examples of the fuel include glucose, fructose, ascorbic acid (vitamin C), alcohol, and lactic acid (see also FIG. 2).

The water absorbent constituting the conductive portion 20 contains a buffer agent as an electrolyte. The buffer agent is an electrolyte that serves as a buffer solution in the form of an aqueous solution. Examples of the buffer agent include salts such as weak acids and weak bases. The water absorbent may or may not contain an electrolyte other than the buffer agent, for example, a salt of a strong acid and a strong base. Examples of the electrolyte constituting the buffer agent include weak acids such as phosphoric acid, acetic acid, citric acid, and tartaric acid; sodium salt, potassium salt, and the like of these weak acids; and weak bases such as organic amines, salts thereof, and the like. The buffer agent may include two or more electrolytes. In a case where the water absorbent does not contain the buffer agent, the buffer agent may be contained in water to be absorbed, or the buffer agent may be contained in both the water absorbent and the water to be absorbed.

The water absorbent of the conductive portion 20 after the transdermal current-carrying patch 1 is manufactured and before the transdermal current-carrying patch is used is in a dry state. When the transdermal current-carrying patch 1 is used, water is supplied to the transdermal current-carrying patch 1, and thus, the water absorbent absorbs the water. As a result, an electrolytic solution containing the electrolyte is contained inside the water absorbent. Accordingly, the anode electrode 11 and the cathode electrode 12 are electrically connected to the skin through the electrolytic solution, and an ion movement path including the anode electrode 11, the conductive portion 20A, the skin, a conductive portion 20B, and the cathode electrode 12 is formed. For example, cations such as hydrogen ions and sodium ions are transported from the anode electrode 11 toward the cathode electrode 12.

In the water absorbent of the conductive portion 20, the buffer agent is contained in a sponge having air bubbles. Examples of a material of the sponge include a synthetic resin such as polyurethane and polyvinyl alcohol; and natural polymers such as cellulose, and derivatives thereof. Fine open air bubbles are formed inside the sponge. Thus, an electrolyte of a solute is in a dry state in the sponge by causing the sponge to absorb the electrolytic solution including the aqueous solution of the electrolyte and then drying the electrolytic solution. It is considered that at least a part of the electrolyte is exposed in a solid state on an inner wall surface of the air bubble without being taken into the material of the sponge. In addition to the electrolyte, the sponge may contain a fuel for a biobattery, medicament that may act on a living body, other additives, and the like.

Since the sponge of the conductive portion 20 is excellent in water absorbency due to capillary phenomenon, surface tension, hydrophilicity, and the like, the sponge quickly absorbs water by merely immersing a part of a lower surface or the like in water. Further, the solute such as the electrolyte is dissolved in water in an inner space of the air bubble of the sponge to prepare the electrolytic solution. Due to water absorption power of the sponge, the electrolytic solution is uniformly mixed and spreads over the entire water absorbent, and the anode electrode 11 and the cathode electrode 12 may be connected to the skin by the electrolytic solution. The water absorbent formed by using the sponge may move moisture even in a direction against gravity or a complicated shape such as a three-dimensional shape.

The sponge constituting the conductive portion 20 has a pore diameter of, for example, 10 to 500 μm. Specific examples of the pore diameter include 10 μm, 20 μm, 25 μm, 30 μm, 50 μm, 80 μm, 100 μm, 150 μm, 200 μm, 300 μm, 500 μm, and the like, or intermediate values and values in the vicinity thereof, and are not limited thereto. A porosity of the sponge is, for example, 60 to 95%. As the sponge, a polyurethane sponge is preferable, and similarly, a sponge having excellent performance such as water absorbency may be suitably used. For example, sofras (product name, manufactured by AION Co., Ltd.) may be used as the sponge constituting the conductive portion 20. Note that, since the sponge constituting the conductive portion 20 has a thickness of about 0.5 mm to 2 mm but has a large number of pores, the thickness may be adjusted when the sponge is incorporated into the transdermal current-carrying patch 1.

In the transdermal current-carrying patch 1 using the biobattery, one or more kinds of enzyme electrodes may be used for the anode electrode 11 or the cathode electrode 12. When the water absorbent of the conductive portion 20 absorbs water, current-carrying of the biobattery is started, and the transdermal current-carrying patch 1 is driven by the biobattery. The water absorbent of the conductive portion 20 allows mass movement of ions, a fuel, and the like between the skin, the anode electrode 11, and the cathode electrode 12 while holding the electrolytic solution like a tank.

The adhesive layer 30 is a member for attaching the transdermal current-carrying patch 1 to the skin of any part of the subject. The adhesive layer 30 may preferably include a double-sided adhesive tape having insulating properties. For example, an acrylic-based adhesive or a silicone-based adhesive can be used as the adhesive layer 30. An adhesive force of the adhesive layer 30 is preferably 1 N/cm or more or 2 N/cm or more, and preferably 20 N/cm or less, 12 N/cm or less, 6 N/cm or less, or 3 N/cm or less. When the adhesive force is too weak, there is a possibility that the transdermal current-carrying patch may be unintentionally peeled off during application. On the other hand, when the adhesive force is too strong, the transdermal current-carrying patch is attached for a long period of time, and there is a possibility that strong stimulation is given to the skin when the patch is peeled off from the skin. Two openings 31 and 32 are provided in the adhesive layer 30, the anode electrode 11 is housed in one opening 31, and the cathode electrode 12 is housed in the other opening 32. The lead 13 between the anode electrode 11 and the cathode electrode 12 is attached onto the portion 33 between the opening 31 and the opening 32. Accordingly, the position of the electrode body 10 with respect to the adhesive layer 30 is fixed. In addition, in the adhesive layer 30, the anode electrode 11 housed in the opening 31 comes into contact with the conductive portion 20A, and the cathode electrode 12 housed in the opening 32 comes into contact with the conductive portion 20B. At this time, outer frame portions of the conductive portions 20A and 20B are also fixed to the adhesive layer 30. With such a configuration, ion insulation is achieved between the conductive portion 20A and the conductive portion 20B. Note that, the adhesive layer 30 has a thickness of, for example, about 0.1 mm to 0.5 mm. The double-sided adhesive tape having insulating properties is used as the adhesive layer 30, and thus, it is possible to downsize the transdermal current-carrying patch 1 while securing both fixation of positions of the electrode body 10 and the conductive portions 20A and 20B with respect to the adhesive layer 30 and ion insulation between the conductive portion 20A and the conductive portion 20B. In particular, the thickness is reduced, and thus, it is possible to facilitate attachment to a joint portion and a local portion.

The separator 40 is a member for achieving the ion insulation between the conductive portion 20A and the conductive portion 20B together with the adhesive layer 30, and may be made of, for example, a film such as polyester or polyethylene terephthalate or release paper in which a surface of paper is coated with silicone or the like. Two openings 41 and 42 are provided in the separator 40, the conductive portion 20A is housed in one opening 41, and the conductive portion 20B is housed in the other opening 42. Note that, the separator 40 has a thickness of, for example, about 0.05 mm to 0.1 mm.

The surface film 50 is a member that covers and protects the electrode body 10 and the conductive portion 20, and may be made of, for example, a polyvinyl chloride film. In a case where oxygen is used as the catalyst, a window portion 51 is formed at a position corresponding to the cathode electrode 12 of the surface film 50 in order to supply the enzyme to the cathode electrode 12. In order to avoid exposure of the cathode electrode 12, the cathode electrode 12 may be protected by using cotton or the like, which is a material capable of transmitting oxygen, for the window portion 51.

The transdermal current-carrying patch 1 having such a configuration may be formed as a small and thin current-carrying patch, and can be easily attached to a predetermined part of the subject for a long time. In the transdermal current-carrying patch 1, when the transdermal current-carrying patch is attached to the predetermined part of the subject after water absorption, the anode electrode 11 and the cathode electrode 12 may come into contact with the living body via the conductive portions 20A and 20B to form the electric circuit that generates the weak current to flow through the predetermined part (including an adjacent region). In the transdermal current-carrying patch 1, the electric circuit is formed by using, as the weak current flowing through the living body, a DC current having a current density of 10 μA/cm² or more in a case where a resistor has 5 kΩ.

Here, an electric resistor in the living body to which the transdermal current-carrying patch 1 is applied will be described. The electric resistor of the living body may be divided into a resistor of the skin and a resistor inside a human body. The resistor of the skin varies depending on a degree of wetting of a contact surface or the like (see Chapter 4 of Occupational Safety and Health Handbook for Electrical Installation, The Ship's Electric Installation Contractors' Association of Japan). When the skin is dry and hard, the skin resistor has about 10 kΩ, but when the skin is sweating, the skin resistor decreases to 1/12. In addition, since the skin resistor when the skin is sweating has about 1 kΩ, the transdermal current-carrying patch 1 according to the present embodiment is desirably formed such that a DC current of 500 μA/cm² or less flows when the transdermal current-carrying patch is connected to a resistor of 1 kΩ. Accordingly, the subject's feeling of stimulation is reduced.

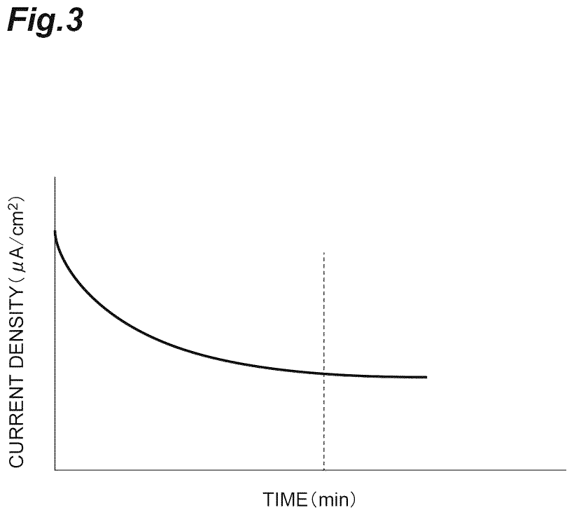

FIG. 3 illustrates a relationship between a current density (μA/cm²) of the current flowing through the electric circuit to be formed by the transdermal current-carrying patch 1 and an elapsed time (minutes). This current density is a current density when the electric circuit of the transdermal current-carrying patch 1 is connected to a resistor of 10 kΩ. In the transdermal current-carrying patch 1, although the current density is slightly high immediately after the start, the current density falls within the above-described range of the weak current with the lapse of time. More specifically, the electric circuit to be formed by the transdermal current-carrying patch 1 is configured to generate a DC current having a current density of 10 μA/cm² or more and 100 μA/cm² at the time of connection to a resistor of 10 kΩ to flow to the predetermined part of the subject. Preferably, the transdermal current-carrying patch 1 is configured such that the weak current flowing through the predetermined part is 10 μA/cm² or more and 175 μA/cm² or less at a point in time when a predetermined time (for example, 10 minutes at the latest) elapses after the electric circuit brings the transdermal current-carrying patch into contact with the predetermined region of the subject. More specifically, the electric circuit of the transdermal current-carrying patch 1 is preferably configured such that the current density of the weak current flowing at a point in time at the latest 10 minutes after being connected to a resistor of 5 kΩ is 10 μA/cm² or more and 175 μA/cm² or less. More preferably, the transdermal current-carrying patch 1 is configured such that the electric circuit maintains the current density of the weak current flowing at 10 μA/cm² or more and 175 μA/cm² or less at a point in time when 5 hours or more elapses after the transdermal current-carrying patch 1 is connected to a resistor of 5 kΩ. That is, it is possible to continuously provide a weak current in a predetermined range by attaching the transdermal current-carrying patch 1 of the present embodiment to the predetermined part of the subject for a long time.

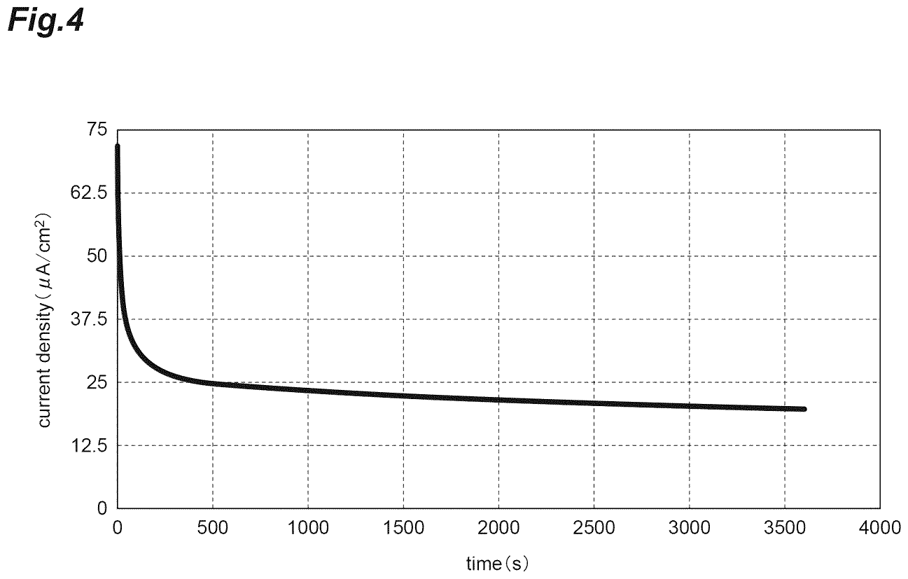

FIG. 4 illustrates an example of the current density of the transdermal current-carrying patch 1. This is a graph of the current density by one sample of the transdermal current-carrying patch 1 actually produced. According to this transdermal current-carrying patch, when the transdermal current-carrying patch is connected to a resistor of 5 kΩ, the current density of the weak current flowing through the predetermined part is in a range of 10 μA/cm² to 30 μA/cm² at a point in time after 10 minutes (600 seconds) elapses, and the weak current flowing through the predetermined region of the subject is maintained in a range of 10 μA/cm² to 30 μA/cm² even at a point in time after 1 hour or more elapses. The kind and amount of the catalyst or electron transfer mediator used in the transdermal current-carrying patch 1 are changed and adjusted, and thus, the DC current flowing through the electric circuit of the transdermal current-carrying patch can be in the above-described range. However, the electric circuit may be configured to generate the DC current having the current density of 35 μA/cm² or more to flow when the electric circuit is connected to a resistor of 5 kΩ, or may be configured to generate the DC current having the current density of 60 μA/cm² or more to flow when the electric circuit is connected to a resistor of 5 kΩ.

The amount of energy generated in the transdermal current-carrying patch 1 is, for example, 5 mJ or more in a case where the transdermal current-carrying patch 1 is connected to a resistor of 10 kΩ for 1 hour. The amount of energy generated in the transdermal current-carrying patch 1 is, for example, 50 mJ or more in a case where the transdermal current-carrying patch 1 is connected to a resistor of 10 kΩ for 10 hours. Note that, the amount of energy generated in the transdermal current-carrying patch 1 when the transdermal current-carrying patch 1 is connected to a resistor of 10 kΩ may be 3600 mJ or less or 5000 mJ or less.

A time during which the DC current flows through the electric circuit of the transdermal current-carrying patch 1, that is, a current-carrying time is, for example, 72 hours or less, 60 hours or less, 48 hours or less, 36 hours or less, 24 hours or less, 1 hour or more, 2 hours or more, 3 hours or more, 4 hours or more, 6 hours or more, 8 hours or more, or 12 hours or more in a case where current carrying is continuously performed. The current-carrying time is, for example, 1 hour to 72 hours, 2 hours to 48 hours, or 3 hours to 24 hours.

### EXAMPLES

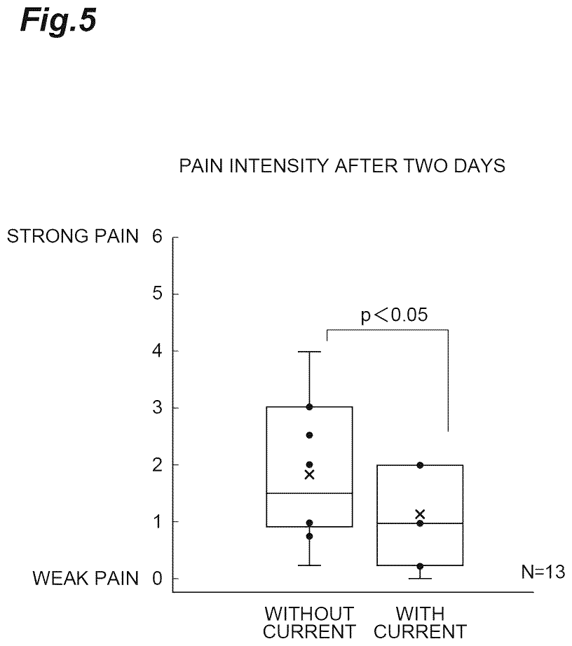

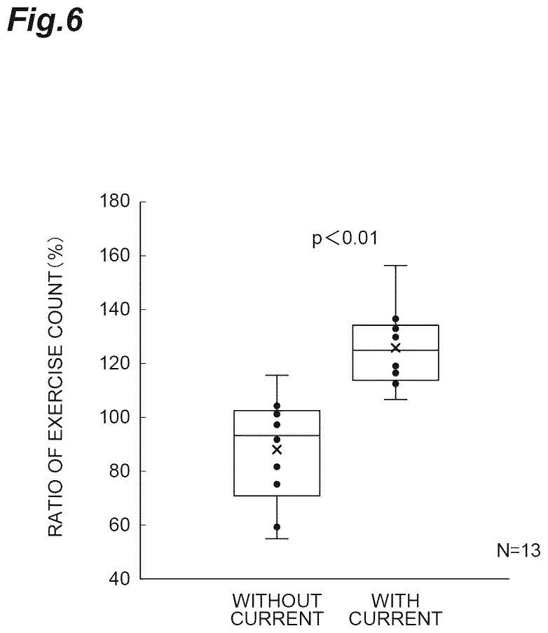

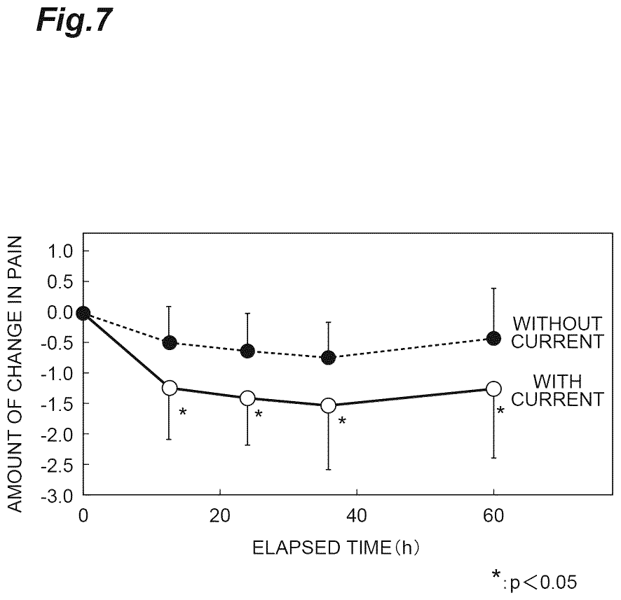

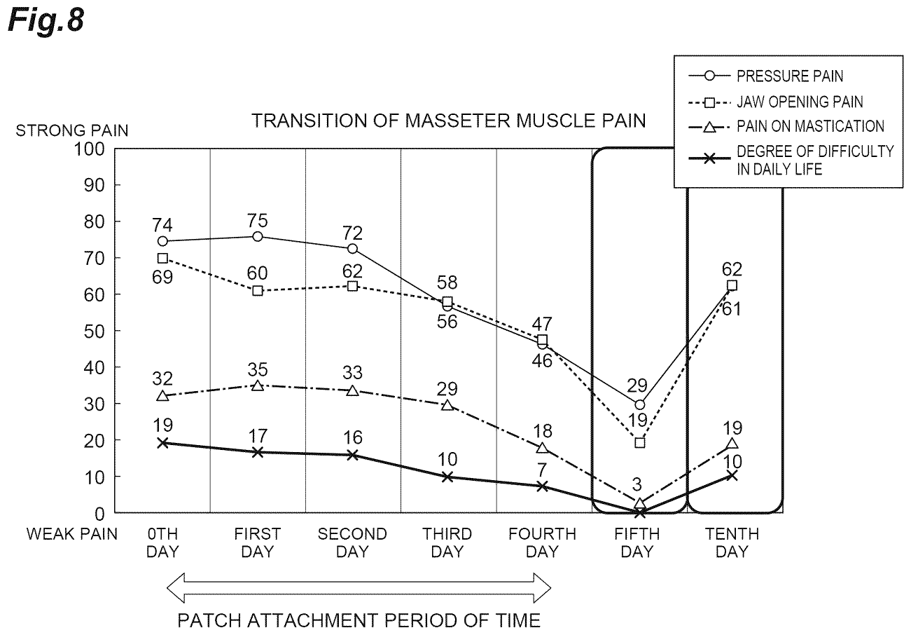

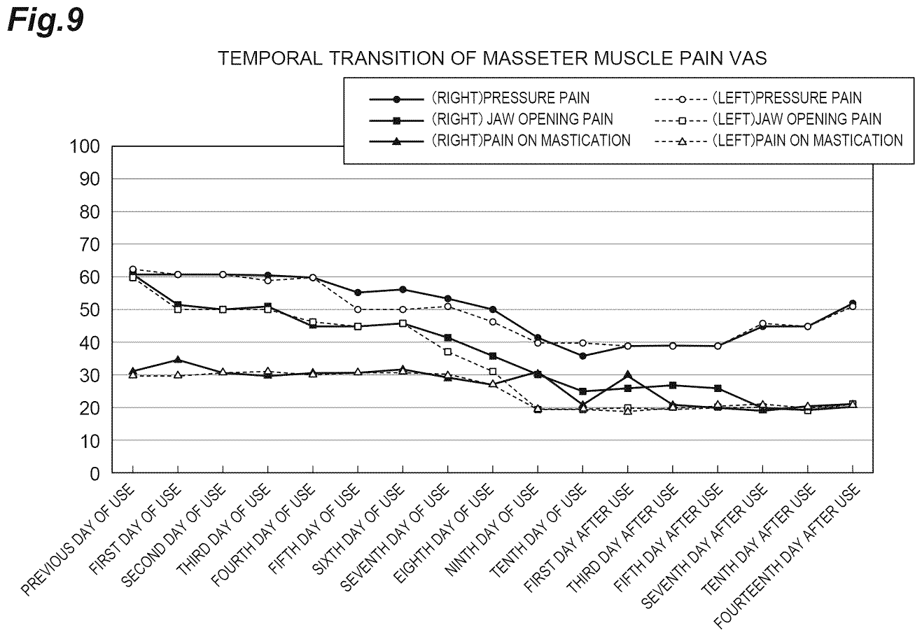

Here, operations and effects obtained by using the transdermal current-carrying patch 1 capable of providing the above-described range of the DC current to the predetermined part of the subject for the subject will be described by using some experimental examples with reference to FIGS. 5 to 9. Experimental Examples 1 to 4 were the following (1) to (4)
(1) Evaluation of delayed onset muscle soreness using transdermal current-carrying patch 1 (see FIG. 5).
(2) Evaluation result of exercise performance using transdermal current-carrying patch 1 (see FIG. 6).
(3) Evaluation result of resolution of stiff shoulder using transdermal current-carrying patch 1 (see FIG. 7).
(4) Evaluation result of resolution of temporomandibular joint disorder using transdermal current-carrying patch 1 (see FIGS. 8 and 9).

First, a large number of transdermal current-carrying patches 1 (first example) used in Experimental Examples (1) to (4) were produced. In the production of the first example of the transdermal current-carrying patch 1, the following materials were prepared.

Electrode body 10: the electrode body 10 having the configuration illustrated in FIG. 1 was produced (prepared) by using carbon fibers (manufactured by Toho Tenax Co., Ltd.) on which multi-walled carbon nanotubes (manufactured by Baytube) were carried as a material. Note that, the carbon nanotube may be manufactured by Meijo Nano Carbon, and is not particularly limited. In addition, the carbon fiber may be manufactured by Toray Industries, Inc., and is not particularly limited. A thickness of the electrode body 10 was 0.3 mm. An area of each of the anode electrode 11 and the cathode electrode 12 was 0.8 cm². 4-isopropylaminodiphenylamine and glucose dehydrogenase were carried, as catalysts, on the anode electrode 11. Carbon fibers on which multi-walled carbon nanotubes and polytetrafluoroethylene were carried were used for the cathode electrode 12. Iron phthalocyanine (manufactured by Tokyo Chemical Industry Co., Ltd.) was carried as a catalyst. The lead 13 was made of the carbon fiber. The anode electrode 11 and the cathode electrode 12 were joined to the lead 13 by thermal adhesion.

Conductive portion 20: the conductive portion 20 was produced (prepared) by adding 300 μL of a 50 mM Mcilvaine buffer solution (pH5) and a 200 mM glucose solution to a sponge (sofras (product name) manufactured by AION Co., Ltd.) made of polyurethane and drying the sponge. A thickness of the conductive portion 20 was 1 mm.

Adhesive layer 30: the adhesive layer 30 was prepared by using a medical double-sided adhesive tape (manufactured by 3M Japan Ltd.) as a double-sided adhesive tape for skin. A thickness of the adhesive layer 30 was 0.16 mm.

Separator 40: the separator 40 having the configuration illustrated in FIG. 1 was produced by using polyester as a material. However, a single-sided polyethylene-coated paper, polypropylene, or the like may be used as the separator 40.

Surface film 50: the separator 40 having the configuration illustrated in FIG. 1 was produced by using a polyvinyl chloride film as a material.

After the above-described materials were prepared, a larger number of first examples of the transdermal current-carrying patch 1 were produced by assembling the electrode body 10, the conductive portion 20, the adhesive layer 30, the separator 40, and the surface film 50 in the order and arrangement illustrated in FIG. 1. The current density by the electric circuit of the patch according to the first example was represented in the following Table 1. The “current density” in Table 1 was a value after about 10 minutes from the addition of the solution containing a substrate, and was a value that slightly decreased after 60 minutes.

TABLE 1

| Resistor R | Voltage | Current density |
|---|---|---|
| (kΩ) | (V) | (μA/cm²) |
| 20 | 0.27 | 13 |
| 10 | 0.23 | 23 |
| 5 | 0.20 | 39 |
| 2 | 0.14 | 72 |
| 1 | 0.11 | 108 |

In the first example of the transdermal current-carrying patch 1, the current density of the weak current flowing when the electric circuit of the patch was connected to a resistor of 10 kΩ was in a range of 10 μA/cm² to 30 μA/cm² at a point in time at the latest 10 minutes after the addition of water to the transdermal current-carrying patch. That is, the patch was configured such that the weak current flowing through the predetermined part of the subject was maintained at 10 μA/cm² or more even at a point in time when 1 hour or more had elapsed. In addition, an open circuit voltage of the transdermal current-carrying patch was about 300 mV. In addition, as represented in Table 1, in the patch of the first example, it was confirmed that a DC current having a current density of 39 μA/cm² at the time of connection of 5 kΩ and a current density of 108 μA/cm² at the time of connection of 1 kΩ flowed. That is, it was confirmed that the current density of the first example of the transdermal current-carrying patch 1 used in the experiment was less than 500 μA/cm², and there was no risk of skin stimulation.

In addition, a second example of the transdermal current-carrying patch 1 was produced. In order to produce the patch according to the second example, first, the following materials were prepared. In the anode electrode 11, 1,4-naphthoquinone (manufactured by Tokyo Chemical Industry Co., Ltd.) was used instead of 4-isopropylaminodiphenylamine used in the first example. A platinum mesh (manufactured by BAS Inc.) was used as the cathode electrode 12, and a stainless steel wire was used as the lead 13. The anode electrode 11 and the cathode electrode 12 were fixed to the lead 13 with an instantaneous adhesive. The other components were assembled as in the first example. Power generation was started by adding a 100 mM potassium phosphate buffer solution (pH7) containing 200 mM glucose to the sponge of the conductive portion 20. The current density by the electric circuit of the patch according to the second example was represented in the following Table 2. The “current density” in Table 2 was a value after about 10 minutes from the addition of the solution containing the substrate, and was a value that slightly decreased after 60 minutes.

TABLE 2

| Resistor R | Voltage | Current density |
|---|---|---|
| (kΩ) | (V) | (μA/cm²) |
| 20 | 0.38 | 24 |
| 10 | 0.34 | 42 |
| 5 | 0.29 | 73 |
| 2 | 0.22 | 135 |
| 1 | 0.15 | 193 |

As in the first example, in the second example of the transdermal current-carrying patch 1, the current density of the weak current flowing when the electric circuit of the patch was connected to a resistor of 10 kΩ was in a range of 20 μA/cm² to 45 μA/cm² at a point in time when 10 minutes had elapsed at the latest from the addition of the substrate. That is, the patch was configured such that the weak current flowing through the predetermined part of the subject was maintained at 20 μA/cm² or more even at a point in time when 1 hour or more had elapsed. In addition, in the patch of the second example, it was confirmed that a DC current having a current density of 73 μA/cm² at the time of connection of 5 kΩ and a current density of 193 μA/cm² at the time of connection of 1 kΩ flows. That is, it was confirmed that the current density of the second example of the transdermal current-carrying patch 1 used in the experiment was less than 500 μA/cm² and there was no risk of skin stimulation.

In addition, a third example of the transdermal current-carrying patch 1 was produced. In order to produce the patch according to the third example, first, the following materials were prepared. Carbon fibers on which multi-walled carbon nanotubes are carried were used for both the anode electrode 11 and the cathode electrode 12. The carbon fibers were electrically connected to an alkaline button battery (1.5 V, LR44, manufactured by Panasonic Corporation). The anode electrode 11 and the cathode electrode 12 were connected by a stainless steel wire (corresponding to the lead 13). The other components were assembled as in the first example of the transdermal current-carrying patch 1. Power generation was started by adding a 100 mM potassium phosphate buffer solution (pH7) to the sponge of the conductive portion 20. The current density by the electric circuit of the patch according to the third example was represented in the following Table 3. The “current density” in Table 3 was a value after about 10 minutes from the connection of the electric circuit, and was a value that slightly decreased after 60 minutes.

TABLE 3

| Resistor R | Voltage | Current density |
|---|---|---|
| (kΩ) | (V) | (μA/cm²) |
| 20 | 1.20 | 60 |
| 10 | 0.97 | 97 |
| 5 | 0.74 | 147 |
| 2 | 0.48 | 242 |
| 1 | 0.41 | 411 |

As in the first example, in the third example of the transdermal current-carrying patch 1, the current density of the weak current flowing when the electric circuit of the patch was connected to a resistor of 10 kΩ was in a range of 70 μA/cm² to 100 μA/cm² at a point in time when 10 minutes had elapsed at the latest from the addition of the substrate. That is, the patch was configured such that the weak current flowing through the predetermined part of the subject was maintained at 50 μA/cm² or more even at a point in time when 1 hour or more had elapsed. In addition, in the patch of the third example, it was confirmed that a current having a current density of 147 μA/cm² at the time of connection of 5 kΩ2 and a current density of 411 μA/cm² at the time of connection of 1 kΩ flows. That is, it was confirmed that the current density of the third example of the transdermal current-carrying patch 1 used in the experiment was less than 500 μA/cm² and there was no risk of skin stimulation.

The amounts of energy generated in the transdermal current-carrying patches according to the first to third examples will be described. As results of calculating the amount of energy generated in the transdermal current-carrying patch when the transdermal current-carrying patch according to the first example is connected to a resistor of 10 kΩ, the amount of energy was 8 mJ in a case where the connection was made for 1 hour and 60 mJ when the connection was made for 10 hours. As results of calculating the amount of energy generated in the transdermal current-carrying patch when the transdermal current-carrying patch according to the second example is connected to a resistor of 10 kΩ, the amount of energy was 30 mJ in a case where the transdermal current-carrying patch was connected for 1 hour. The energy amount in a case where the transdermal current-carrying patch according to the second example is connected for 10 hours is estimated to be 225 mJ from the calculation result of the transdermal current-carrying patch according to the first example. As a result of calculating the amount of energy generated in the transdermal current-carrying patch when the transdermal current-carrying patch according to the third example is connected to a resistor of 10 kΩ, the amount of energy was 480 mJ in a case where the transdermal current-carrying patch was connected for 1 hour. The amount of energy in a case where the transdermal current-carrying patch according to the third example is connected for 10 hours is estimated to be 3600 mJ from the calculation result of the transdermal current-carrying patch according to the first example.

### Experimental Example 1

In Experimental Example 1, delayed onset muscle soreness using the transdermal current-carrying patch 1 (first example) was evaluated. In addition, in the transdermal current-carrying patch 1 (first example), a negative control product in which electricity excluding glucose dehydrogenase, iron phthalocyanine, and the like involved in the delivery and acceptance of electrons did not flow was also used for the experiment. In this experiment, a dumbbell (male: 7.5 kg, female: 5 kg) was held in a state where an elbow is placed on a pedestal, and an exercise of lifting the dumbbell at one reciprocation/4 seconds and then returning the dumbbell to an initial position was repeated twice in a row until pace could not be maintained. There were 13 subjects (6 males, 7 females). After the dumbbell exercise was completed, the produced transdermal current-carrying patch 1 or the negative control product was attached to a long muscle of biceps brachii muscle of the subject such that a current flows along muscle fibers, and a weak current having a current density in a range of 10 μA/cm² to 30 μA/cm² flowed continuously through a predetermined part of the subject. This experiment was performed in a state where the subject does not know which of the transdermal current-carrying patch 1 and the negative control product was attached. The weak current was provided over 12 hours from the lapse of one day after the exercise, and further over 12 hours from the lapse of one day to the lapse of two days. All 12 hours included the bedtime. In addition, the subject again performed the same dumbbell experiment at an interval of at least two weeks after the above-described dumbbell exercise was completed. In a first dumbbell experiment, the experiment was performed as in the experiment in which the negative control product was attached to the subject to whom the transdermal current-carrying patch 1 was attached. On the other hand, the experiment was performed as in the first dumbbell experiment in which the transdermal current-carrying patch 1 was attached to the subject to whom the negative control product was attached.

Two days after completion of the dumbbell exercise, each subject was evaluated for a condition of delayed onset muscle soreness (a type of muscle pain). The Japanese version of Talag scale was used, and evaluation criteria were as follows (24 steps of 0.25 steps between 0 and 6). (see the Society of Physical Therapeutic Science, 22(1), 125-131 (2007))
0: No pain.
1: Discomfort
2: Slightly distinct pain
3: A little more distinct pain
4: Distinct pain
5: Strong pain
6: Unbearable pain.

FIG. 5 illustrates results of pain intensity after two days. As illustrated in FIG. 5, it was confirmed that pain intensity when current-carrying processing is performed for 12 hours×twice after the dumbbell exercise by using the transdermal current-carrying patch 1 (first example, with current) was lower than pain intensity when the current-carrying processing is not performed by attaching the negative control product (without current). A p value was calculated by a sign assignment test of Wilcoxon, and it was also confirmed that p<0.05. In this manner, according to Experimental Example 1 using the patch of the first example, it was confirmed that the pain at the predetermined part can be relieved by causing the weak current having the current density in a range of 10 μA/cm² to 30 μA/cm² to flow through the predetermined part. It is considered that the muscle pain is not superficial pain that is pain on a skin surface but deep pain derived from muscles that are internal tissues. Thus, it cannot be assumed that the pain of the muscle pain is alleviated by merely causing a slight current to flow through the skin surface for a short period of time. It is considered that the pain alleviation of the muscle pain that is deep pain is achieved by causing the weak current to continuously flow by using the transdermal current-carrying patch 1.

### Experimental Example 2

In Experimental Example 2, exercise performance using the transdermal current-carrying patch 1 (first example) was evaluated. In this experiment, the same dumbbell exercise as in Experimental Example 1 was performed, and the number of dumbbells for the first dumbbell exercise was counted. The number of subjects was 13 as in Experimental Example 1. As in Experimental Example 1, after the dumbbell exercise was completed, the above-described transdermal current-carrying patch 1 (first example) or the negative control product was attached to biceps brachii muscle for 12 hours (12 hours×twice) on each of a first day and a second day. Experimental Example 2 was performed in a state where the subject did not know which of the transdermal current-carrying patch 1 and the negative control product was attached. When the transdermal current-carrying patch 1 was attached, the weak current having the current density in a range of 10 μA/cm² to 30 μA/cm² continuously flowed through the predetermined part of the subject. On the other hand, when the negative control product was attached, processing of causing the weak current having the current density in a range of 10 μA/cm² to 30 μA/cm² was not performed.

Three days after the first dumbbell exercise, all of the subjects performed the same dumbbell exercise as the first dumbbell exercise, continued until arms were not raised, and the number of dumbbells of a second dumbbell exercise was counted. Thereafter, a ratio of “the number of dumbbells of the second dumbbell exercise/the number of dumbbells of the first dumbbell exercise” was calculated as a ratio of exercise count (%) for each subject. As in the first example, the same experiment was performed again after a period of two weeks or longer without applying a load to the biceps brachii muscle. At this time, the negative control product was attached to the subject to whom the transdermal current-carrying patch 1 was attached in a previous dumbbell experiment, and the experiment was performed in the same manner. On the other hand, the transdermal current-carrying patch 1 (first example) was attached to the subject to whom the negative control product was attached in the previous dumbbell experiment, and the experiment was performed in the same manner. FIG. 6 illustrates the ratio of exercise count calculated in this manner divided into a first group (with current-carrying processing) and a second group (without current-carrying processing). As illustrated in FIG. 6, it was confirmed that the number of times of lifting the dumbbell was improved in the first group in which the current-carrying processing was performed for 12 hours×twice after the dumbbell exercise by using the transdermal current-carrying patch 1 (first example). A p value was calculated by a sign assignment test of Wilcoxon, and it was also confirmed that p<0.01. In this manner, according to Experimental Example 2 using the patch of the first example, it was confirmed that exercise capability was improved by continuously applying the weak current having the current density in a range of 10 μA/cm² to 30 μA/cm². It was surprising that the exercise capability can be improved by influencing muscles which are internal tissues by causing the weak current to flow.

Next, the same experiment as in Experimental Example 2 described above was performed by using the patch of the second example instead of the patch of the first example. However, an attachment time was 1 hour on the first day and 4 hours on the second day. A target was a male in 40s (one person). As a result, the ratio of exercise count (%) when the negative control product was attached was 46%. On the other hand, the ratio of exercise count (%) when the patch of the second example was attached was 152%. Accordingly, it was confirmed that exercise capability was improved by continuously applying the weak current having the current density in a range of 20 μA/cm² to 45 μA/cm². Subsequently, the same experiment as in Experimental Example 2 described above was performed by using the patch of the third example instead of the patch of the first example. However, the attachment time was set to 4 hours on the first day and 4 hours on the second day. A target was a male in 30s (one person). As a result, the ratio of exercise count (%) when the negative control product was attached was 88%. On the other hand, the ratio of exercise count (%) when the patch of the third example was attached was 108%. Accordingly, it was confirmed that exercise capability was improved by continuously applying the weak current having the current density in a range of 70 μA/cm² to 100 μA/cm². In addition, as a result of evaluating the pain after two days, the pain at the time of attaching the negative control product was evaluated as “3”, whereas the pain at the time of attaching the patch of the third example was evaluated as “0”. That is, it was confirmed that a pain reduction effect is obtained by causing the above-described current to flow.

### Experimental Example 3

In Experimental Example 3, the resolution of stiff shoulder using the transdermal current-carrying patch 1 was evaluated. In this experiment, the above-described transdermal current-carrying patch 1 (first example) was attached to each of the pain parts on the shoulders of the subjects (15 persons) in the first group for 12 hours, and the weak current having the current density in a range of 10 μA/cm² to 30 μA/cm² was continuously applied to each of the predetermined parts of the subjects. Then, reduction of pain after a certain time (after 12 hours, after 24 hours, after 36 hours, after 60 hours) was investigated. On the other hand, the negative control product was attached to each of the pain parts on the shoulders of the subjects (15 persons) in the second group, and reduction of pain only by natural healing over time was investigated. The subject did not know which patch was attached. The evaluation criteria were the same as in Experimental Example 1, and how much the pain changed from the pain before attachment was recorded. The experiment results are illustrated in FIG. 7. As illustrated in FIG. 7, it was confirmed that pain was relieved in the first group in which the current-carrying processing was performed on the pain part of the shoulder for 12 hours by using the transdermal current-carrying patch 1 (first example) as compared with the second group in which the current-carrying processing was not performed.

As a comparative example, the amount of change in pain was recorded in the same manner by using a general magnetic therapeutic device. There were 12 subjects. The general magnetic therapeutic device was attached for three consecutive days. As a result, the amount of change in pain was −1 after 24 hours, −0.9 after 36 hours, and −0.79 after 60 hours from the start of the attachment of the general magnetic therapeutic device, and the pain tended to be relieved. However, the use of the transdermal current-carrying patch (first example) resulted in a higher pain reduction effect. Note that, the transdermal current-carrying patch (first example) had a shorter attachment time than in the comparative example, and had a higher reduction effect.

### Experimental Example 4

In Experimental Example 4, a degree of pain resolution of temporomandibular joint disorder using the transdermal current-carrying patch 1 (first example) was evaluated. In this experiment, for one subject (30s, female) for which the temporomandibular joint disorder was diagnosed, the above-described transdermal current-carrying patch 1 was attached to the pain part of the temporomandibular joint for 4 days such that a current flowed along muscle fibers of masseter muscle at bedtime, and a weak current having a current density in a range of 10 μA/cm² to 30 μA/cm² flowed continuously through the predetermined part of the subject. Then, by using the visual analogue scale (VAS) every morning, a degree of improvement in pain was evaluated in a range of intensity 0 (no pain) to 100 (most painful). For the degree of improvement in pain, pressure pain, jaw opening pain, pain on mastication, and a degree of difficulty in daily life were evaluated. The pressure pain indicates muscle pain when muscle part pressurization is applied to the jaw at a pressure of 1 kg, the jaw opening pain indicates jaw pain when the mouth is opened, and the pain on mastication indicates jaw pain when food is chewed. The degree of difficulty in daily life is a standard of how much the jaw pain interferes with daily life, and was evaluated in a range of 0 (without difficulty) to 100 (with difficulty as bad as it gets).

Here, the temporomandibular joint disorder will be described. The temporomandibular joint disorder is a third dental disease that is comparable to dental caries and periodontal disease. The number of patients with any symptom of a temporomandibular joint is estimated to be about 19 million in Japan. It is recommended that a first choice of treatment for the temporomandibular joint disorder is a conservative, reversible and evidence-based treatment (see guidelines for treatment of temporomandibular joint disorder 2020, The Japanese Society for Temporomandibular Joint). A basic treatment of masticatory myalgia disorder (type I) that is a most common form of temporomandibular joint disorder is based on physical therapy. Not only patients who develop only type I, but also patients who are complicated with temporomandibular joint pain disorder (type II), temporomandibular joint disc disorder (type III), and deformability temporomandibular joint disorder (type IV) are included. Specifically, there are self-massage of an affected part, a warm fomentation method of warming the affected part, and a pain relief therapy (transcutaneous electrostimulation therapy) by electrical stimulation. The transcutaneous electrostimulation therapy is said to cause contraction and relaxation of muscles by electrical stimulation to alleviate hypertonia. However, some cases cannot be sufficiently treated by these physical therapies. There is also a systematic review describing that the treatment using the transcutaneous electrostimulation therapy failed to show sufficient effectiveness (T. List, S. Axelsson, Journal of Oral Rehabilitation (2010)). Accordingly, there is a demand for physical therapy capable of more effective treatment and pain alleviation.

Electric therapeutic devices other than the transcutaneous electrostimulation therapy have been applied only to a part below the neck in the living body, and the effectiveness of treatment of the temporomandibular joint disorder has been unknown. In particular, a report example in which a weak DC current was applied to temporomandibular joint disorder has not been found, and it has not been clear how much current flows to perform treatment.

FIG. 8 is a table representing a degree of improvement in the temporomandibular joint disorder in Experimental Example 4. As illustrated in FIG. 8, it was confirmed that the pain of the temporomandibular joint disorder that was difficult to be treated could be significantly improved by attaching the transdermal current-carrying patch 1 to the pain part of the jaw continuously for four days during sleeping and causing the weak current to continuously flow in the above-described range. In particular, on a fifth day, it was confirmed that the change was dramatically improved.

Another experiment was performed on one subject (60s, female) for which temporomandibular joint disorder was diagnosed as described above. Although this subject developed type-I, type-II, type-III, and type-IV temporomandibular joint disorders, and performs self-massage and transcutaneous electrostimulation therapy that were normal physical therapies, the pain could not be alleviated. As described above, the transdermal current-carrying patch 1 (first example) was attached during sleeping. However, the patch was attached once a day for two weeks. As a result, as illustrated in FIG. 9, pain alleviation was observed from the fifth day of use for pressure pain and from the first day of use for jaw opening pain. As for the jaw opening pain, the pain alleviation continued even after two weeks from the use, and a higher effect than the existing therapy was recognized. In addition, the transdermal current-carrying patch 1 used in this experiment had a width of 2 cm and a length of 5 cm. In some subjects, pain may occur in a wider range, and a size of the patch is 1 cm or more in width, preferably 3 cm or more in width, and more preferably 4 cm or more in width. A length of the patch is 1 cm or more, preferably 3 cm or more, more preferably 4 cm or more, 5 cm or more, or 6 cm or more. As a result of examining an area of the current patch, it has been found that it is desirable that the area is 50 cm² or less, preferably 40 cm² or less, and more preferably 30 cm² or less in order to attach the current patch to the pain part. In addition, a patch of 1 cm² or more, preferably 5 cm² or more is desirable, and a plurality of small patches may be attached and adjusted to have an appropriate area.

It is considered that all of type I, type II, type III, and type IV temporomandibular joint disorders are not superficial pain that is pain on the skin surface but deep pain derived from muscles and bones that are internal tissues. Thus, it cannot be assumed that the pain of the temporomandibular joint disorder is alleviated by merely causing a slight current to flow through the skin surface for a short period of time. In Experimental Example 4, it is considered that the pain alleviation of the temporomandibular joint disorder that is deep pain is achieved by causing the weak current to continuously flow by using the transdermal current-carrying patch 1.

Note that, in Experimental Examples 1 to 4 (first example) described above, the weak current (current density) provided to the subject was in a range of 10 μA/cm² to 30 μA/cm². On the other hand, in a case where a slightly higher weak current (20 to 45 μA/cm²) in which an exercise performance improving effect was observed was used (second example), it is considered that a cell damage recovery effect was higher. Accordingly, in the treatment of the temporomandibular joint disorder, a current having a current density of 20 to 45 A/cm² (at the time of connection of a 10-kΩ resistor) is carried by using the patch of the second example, and thus, a remedial effect similar to or more than the above remedial effect is also expected.

The weak current flowing through the living body by using the transdermal current-carrying patch 1 is the DC current, and thus, it is assumed that a moving speed of a cell is increased as compared with the case of an AC current. As a result, it is assumed that a repair speed of the cell is increased.

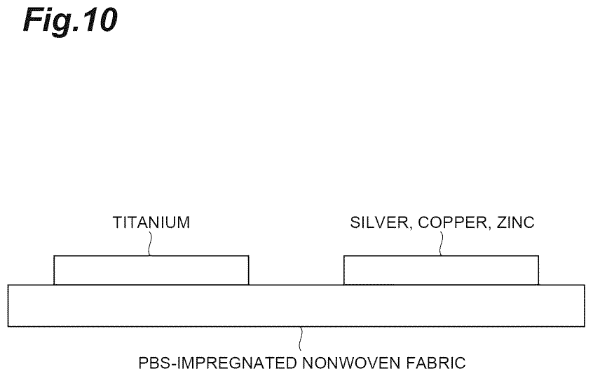

In addition, the current density of the patch in the example of Japanese Unexamined Patent Publication No. 2016-144634 was verified. FIG. 10 is a diagram illustrating an experiment method used for verification. Verification results were as follows. There were the following three kinds of metal batteries in the prior art.
1) Titanium and Silver
2) Titanium and Copper
3) Titanium and Zinc

In this verification method, a nonwoven fabric was infiltrated with 10 mL of physiological saline (PBS), and as illustrated in FIG. 10, two metal electrodes were installed on the nonwoven fabric to form an electric circuit electrically connected. Such a configuration imitates an actual application (attachment to the living body). A current value of each of such electric circuits was measured. Measurement results were represented in the following Table 4. That is, a current density of a current flowing through a current-carrying patch of the prior art is smaller than 0.5 μA/cm².

TABLE 4

| | Resistor R | Voltage | Current density |
|---|---|---|---|
| Kind | (kΩ) | (mV) | (μA/cm²) |
| Titanium and | 5 | 0.82 | 0.21 |
| silver | 10 | 0.10 | 0.13 |
| Titanium and | 5 | 1.6 | 0.15 |
| copper | 10 | 1.1 | 0.22 |
| Titanium and | 5 | 2.0 | 0.4 |
| zinc | 10 | 3.3 | 0.33 |

Note that, in the verification method in which titanium and zinc electrodes were immersed in the physiological saline and were electrically connected while being stirred, it was confirmed that a current of 700 mV and 0.7 μA/cm² flows when the current-carrying patch is connected to a resistor of 1000 kΩ. Thus, in a case where a current of 650 μA described in the example of Japanese Unexamined Patent Publication No. 2016-144634 flows, an electrode area of 100 cm² or more is required as a sectional area through which the current flows, and it has been confirmed that a structure is very large.

As described above, according to the transdermal current-carrying patch 1 of the present embodiment, the anode electrode 11 and the cathode electrode 12 are brought into contact with the part of the subject via the conductive portions 20A and 20B to form the electric circuit that generates the weak current to flow in the part, and the weak current flowing in the living body by the electric circuit is a DC current of 0.5 μA/cm² or more and less than 500 μA/cm². According to the findings of the present inventor, as described above, it has been found that remedial effect on the target part can be significantly improved by setting the weak current flowing through the living body to a DC current having a current density of 0.5 μA/cm² or more, which is slightly higher than a very small amount of current (0.2 μmA/cm² or less). Accordingly, according to the transdermal current-carrying patch 1, the remedial effect of the target part can be improved. In addition, when the current density of the current flowing through the living body is 500 μA/cm² or more, the user may feel stimulation. Thus, in the transdermal current-carrying patch 1, the electric circuit is formed such that the current density of the current flowing through the living body is less than 500 μA/cm². Accordingly, the transdermal current-carrying patch 1 can be used for a long period of time (can be attached to the predetermined part of the user), and the remedial effect of the target part can be further improved.

In addition, in the transdermal current-carrying patch 1 according to the present embodiment, the electric circuit to be formed is configured to generate a DC current having a current density of 10 μA/cm² or more to flow when the transdermal current-carrying patch is connected to a resistor of 5 kΩ. Accordingly, the remedial effect on the target part can be more reliably improved. Note that, the electric circuit may be configured to generate a DC current having a current density of 35 μA/cm² or more to flow when the electric circuit is connected to a resistor of 5 kΩ, or may be configured to generate a DC current having a current density of 60 μA/cm² or more to flow.

In addition, the transdermal current-carrying patch 1 according to the present embodiment is configured to generate a DC current having a current density of less than 500 μA/cm² to flow when the transdermal current-carrying patch is connected to a resistor of 1 kΩ. Accordingly, it is possible to prevent the user from feeling stimulation regardless of the skin condition, and it is possible to more reliably use the transdermal current-carrying patch for a long period of time. Accordingly, it is possible to further improve the remedial effect of the target part.

In addition, in the transdermal current-carrying patch 1 according to the present embodiment, the electric circuit to be formed is configured such that the current density of the weak current flowing at a point in time at the latest 10 minutes after being connected to a resistor of 5 kΩ is 10 μA/cm² or more and 175 μA/cm² or less. Accordingly, it is possible to continuously improve the remedial effect of the target part by attaching the transdermal current-carrying patch 1 to the target part for a long period of time.

In addition, in the transdermal current-carrying patch 1 according to the present embodiment, the electric circuit to be formed is configured to generate a DC current having a current density of 10 μA/cm² or more and 30 μA/cm² or less to flow when the electric circuit is connected to a resistor of 10 kΩ. Accordingly, the remedial effect on the target part can be more reliably improved.

In addition, in the transdermal current-carrying patch 1 according to the present embodiment, the electric circuit to be formed is configured to generate the amount of energy of 50 mJ or more in the electric circuit when the electric circuit is connected to a resistor of 10 kΩ. Accordingly, the remedial effect of the target part can be further improved.

In addition, in the transdermal current-carrying patch 1 according to the present embodiment, the conductive portion 20 includes the conductive portions 20A and 20B corresponding to the anode electrode 11 and the cathode electrode 12, respectively, each of the conductive portions 20A and 20B includes the sponge having the air bubble and the buffer agent made of the electrolyte, and a solid of the buffer agent is exposed on the inner wall surface of the air bubble. In addition, at least one of the anode electrode 11 and the cathode electrode 12 carries the enzyme that catalyzes an oxidation-reduction reaction. Further, the electron transfer mediator 15 is fixed to the electrode (for example, anode electrode 11) that carries the enzyme, and the electron transfer mediator 15 is a mediator of the quinone-based compound or the phenylenediamine-based compound. According to such a configuration, it is possible to more reliably realize the setting of the weak current flowing through the living body to any of the above-described ranges, and to more reliably improve the remedial effect of the target part.

In addition, in the transdermal current-carrying patch 1 according to the present embodiment, the area of each electrode of the anode electrode 11 and the cathode electrode 12 may be 80 cm² or less. In this case, the transdermal current-carrying patch 1 can be downsized, and the transdermal current-carrying patch 1 can be easily attached to the target part of the user for a long period of time. Accordingly, the remedial effect of the target part can be further improved.

In addition, the transdermal current-carrying patch 1 according to the present embodiment includes the double-sided adhesive tape having the insulating properties in which the opening 31 for housing the anode electrode 11 and the opening 32 for housing the cathode electrode 12 are provided, the lead 13 is fixed to one surface of the double-sided adhesive tape, and the conductive portions 20A and 20B are fixed to the other surface of the double-sided adhesive tape. Accordingly, it is possible to downsize the transdermal current-carrying patch 1 while securing both fixation of the positions of the anode electrode 11, the cathode electrode 12, and the conductive portions 20A and 20B and ion insulation between the conductive portion 20A and the conductive portion 20B.

Although the transdermal current-carrying patch 1 according to the present embodiment has been described above, the present invention is not limited to the above embodiment, and various modifications can be applied. For example, in the above embodiment, although it has been described that the biobattery is used, a patch having another configuration may be used as long as the electric circuit that generates the weak current to flow through the part of the subject which is the living body is formed, and the weak current flowing through the part of the subject by the electric circuit is a DC current having a current density of 0.5 μA/cm² or more. For example, a current-carrying patch having a configuration using the button battery illustrated in the third example described above may be used, or a thin film battery may be used instead of the button battery. However, in order to attach the transdermal current-carrying patch to the part of the subject for a long period of time, a small and thin transdermal current-carrying patch is preferable.

The transdermal current-carrying patch may include a diode and means for supplying power by wireless communication. Examples of the means include means described in the following reference. In such a transdermal current-carrying patch, the diode and the means are combined, and thus, a potential difference between the anode electrode and the cathode electrode, that is, a voltage may be a sine wave that fluctuates only in a region of a voltage of 0 V or more or a sine wave that fluctuates only in a region of a voltage of 0 V or less. It should be noted that the voltage does not vary across a region of a positive voltage and a region of a negative voltage. The current-carrying time in such a transdermal current-carrying patch is, for example, the same as the current-carrying time in a case where a DC current flows through the transdermal current-carrying patch. In addition, a frequency is, for example, 0.1 to 200 kHz, 1 to 100 kHz, or 5 to 80 KHz.

Reference: Jiang, Y., Trotsyuk, A. A., Niu, S. et al. Wireless, closed-loop, smart bandage with integrated sensors and stimulators for advanced wound care and accelerated healing. Nat Biotechnol 41, 652-662 (2023). https://doi.org/10.1038/s41587-022-01528-3

The transdermal current-carrying patch may include a DC and pulse converter. In such a transdermal current-carrying patch, a pulse current flows by applying a pulse direct voltage. The pulse current may be an intermittent pulse that does not flow in a case where the voltage is 0 V but flows in a case where the voltage is a positive value. The pulse current may be an intermittent pulse that does not flow in a case where the voltage is 0 V but flows in a case where the voltage is a negative value. The pulse direct voltage is a voltage that fluctuates only in a region of a voltage of 0 V or more or a voltage that fluctuates only in a region of a voltage of 0 V or less. It should be noted that the pulse direct voltage does not vary across the positive voltage region and the negative voltage region. The frequency of the pulse current is, for example, 0.1 to 200 kHz, 1 to 100 kHz, or 5 to 80 kHz. An on and off ratio of the pulse direct voltage is, for example, 1/10 to 20, 1/50 to 15, or 1/30 to 10. The current-carrying time in such a transdermal current-carrying patch is, for example, the same as the current-carrying time in a case where a DC current flows through the transdermal current-carrying patch.

### REFERENCE SIGNS LIST

1 transdermal current-carrying patch
10 electrode body (a plurality of electrodes)
11 anode electrode (negative electrode)
12 cathode electrode (positive electrode)
13 lead (connection portion)
14 enzyme
15 electron transfer mediator
20, 20A, 20B conductive portion (conductive layer, a plurality of conductive portions)

1. A transdermal current-carrying patch comprising:
a positive electrode and a negative electrode; and
a conductive portion disposed to come into contact with the positive electrode and the negative electrode to correspond to the positive electrode and the negative electrode,
wherein, in the transdermal current-carrying patch, an electric circuit that generates a weak current to flow through a living body by bringing the positive electrode and the negative electrode into contact with the living body via the conductive portion is formed, and the weak current flowing through the living body by the electric circuit is a DC current having a current density of 0.5 μA/cm² or more and less than 500 μA/cm².

2. The transdermal current-carrying patch according to claim 1, wherein the electric circuit is configured to generate a DC current having a current density of 10 μA/cm² or more to flow when the electric circuit is connected to a resistor of 5 kΩ.

3. The transdermal current-carrying patch according to claim 1, wherein the electric circuit is configured to generate a DC current having a current density of 35 μA/cm² or more to flow when the electric circuit is connected to a resistor of 5 kΩ.

4. The transdermal current-carrying patch according to claim 1, wherein the electric circuit is configured to generate a DC current having a current density of 60 μA/cm² or more to flow when the electric circuit is connected to a resistor of 5 kΩ.

5. The transdermal current-carrying patch according to claim 1, wherein the electric circuit is configured to generate a DC current having a current density of less than 500 μA/cm² to flow when the electric circuit is connected to a resistor of 1 kΩ.

6. The transdermal current-carrying patch according to claim 1, wherein the electric circuit is configured such that a current density of a weak current flowing at a point in time when 10 minutes elapses at the latest after the electric circuit is connected to a resistor of 5 kΩ is 10 μA/cm² or more and 175 μA/cm² or less.

7. The transdermal current-carrying patch according to claim 1, wherein the electric circuit is configured to generate a DC current having a current density of 10 μA/cm² or more and 30 μA/cm² or less to flow when the electric circuit is connected to a resistor of 10 kΩ.

8. The transdermal current-carrying patch according to claim 1, wherein the electric circuit is configured to generate the amount of energy of 50 mJ or more in the electric circuit when the electric circuit is connected to a resistor of 10 kΩ.

9. The transdermal current-carrying patch according to claim 1, further comprising:
a connection portion configured to electrically connect the positive electrode and the negative electrode to each other,
wherein the conductive portion includes a plurality of conductive portions, respectively, corresponding to the positive electrode and the negative electrode, each of the plurality of conductive portions includes a sponge having an air bubble and a buffer agent made of an electrolyte, and a solid of the buffer agent is exposed on an inner wall surface of the air bubble, and
at least one of the positive electrode and the negative electrode carries an enzyme that catalyzes an oxidation-reduction reaction.

10. The transdermal current-carrying patch according to claim 9, wherein an electron transfer mediator is fixed to the electrode that carries the enzyme.

11. The transdermal current-carrying patch according to claim 10, wherein the electron transfer mediator is a mediator of a quinone-based compound or a phenylenediamine-based compound.

12. The transdermal current-carrying patch according to claim 1, wherein an area of each of the positive electrode and the negative electrode is 80 cm² or less.

13. The transdermal current-carrying patch according to claim 9, further comprising:
a double-sided adhesive tape that has openings provided to house the positive electrode and the negative electrode, and has insulating properties,
wherein the connection portion is fixed to one surface of the double-sided adhesive tape, and
the conductive portion is fixed to the other surface of the double-sided adhesive tape.

14. An operation method for causing a weak current to flow through the living body by using the transdermal current-carrying patch according to claim 1.
